(19) United States
(12) Patent Application Publication
(10) Pub. No.: US 2021/0289963 A1
(43) Pub. Date: Sep. 23, 2021

(54) HERBAL PILLOW

(71) Applicant: HOPPY DREAMS SLEEP COMPANY, LLC, Lake Toxaway, NC (US)

(72) Inventors: Kristi A. Reagor, Charleston, SC (US); Alan James Wilson, Lake Toxaway, NC (US); Cynthia Graham Wilson, Lake Toxaway, NC (US)

(73) Assignee: HOPPY DREAMS SLEEP COMPANY, LLC, Lake Toxaway, NC (US)

(21) Appl. No.: 17/204,198

(22) Filed: Mar. 17, 2021

Related U.S. Application Data
(60) Provisional application No. 62/990,648, filed on Mar. 17, 2020.

(51) Int. Cl. A47G 9/10; A61M 21/02; A47G 9/02

(52) U.S. Cl. CPC A47G 9/1081; A61M 21/02; A61M 2021/0016; A47G 9/0253; A47G 9/10

(57) ABSTRACT

A pillow according to the present disclosure includes a liner that forms an enclosure and a fill that is located within the enclosure, the fill deforming to provide support when the pillow is used. The pillow further includes an herbal mixture, located within the enclosure of the liner, the herbal mixture providing an aroma to the user when the pillow is used.

### RELATED APPLICATION

This application claims priority to U.S. Provisional Patent Application No. 62/990,648, filed on Mar. 17, 2020, the entire contents of such application being fully incorporated herein by reference.

### BACKGROUND

A good night's sleep can be hard to get. Sleep deprivation is a common problem all over the world. In the United States alone, it is estimated that up to thirty-five percent of all adults do not get enough sleep at night. Lack of sleep affects the mood and other characteristics of an individual, and is estimated to cost the United States over $411 billion dollars in lost productivity, resulting in an estimated loss of 1.2 million working days per year.

Many people have difficulty falling asleep while others cannot stay asleep. Many treatments exist for sleep deprivation. For instance, sleep medications are available, but these can be expensive, can lead to addiction, can result in people feeling groggy when they wake up, etc. Therefore, medication has its drawbacks. Other remedies exist, such as taking supplements like cannabidiol and others. However, these can be expensive, can have side effects, etc. What is needed is a safe, natural way to help relieve the sleep deprivation problem.

### SUMMARY

According to an implementation, described herein, a pillow includes a liner that forms an enclosure and a fill that is located within the enclosure of the liner, the fill deforming to provide support when the pillow is used. The pillow further includes an herbal mixture, located within the enclosure of the liner, the herbal mixture providing an aroma to the user when the pillow is used. The herbal mixture and fill may be mixed together within the enclosure of the liner. An envelope may be connected to an inner surface of the liner, the envelope providing a second enclosure, and a sachet may be located within the second enclosure, the herbal mixture located within the sachet. The envelope may be connected to an inner surface of the liner. The envelope may be formed from two flaps of material and a portion of the liner adjacent to the two flaps of material. The envelope may include a closure. The sachet may include a sachet shell configured to allow the aroma from the herbal mixture to be dispersed from the sachet while maintaining the herbal mixture within the sachet. The sachet may include a sachet closure. The pillow may include a pillowcase, the liner being disposed within the pillowcase. The herbal mixture may include lavender, jasmine, chamomile, lemongrass and rose petals. The herbal mixture may include hops and at least one of mugwort, jasmine, chamomile, lavender, rose petals, lemongrass or mint. The herbal mixture may include cedar, rosemary and lavender. The herbal mixture may include cedar, rosemary, lemongrass and thyme. The liner may be formed in a circular shape. The sachet may be disposable. The pillowcase may be formed from canvas, and liner may be disposed within the pillowcase. The liner may be formed in the shape of a neck bolster. The sachet may have at least two compartments, each compartment including the herbal mixture. The sachet may have exactly three compartments. The herbal mixture in each compartment may be composed of the same materials.

### BRIEF DESCRIPTION OF THE DRAWINGS

FIG. 1A represents an example environment in which the technology, described herein, may be implemented.

FIG. 1B illustrates an example embodiment of the sachet depicted in FIG. 1A.

FIG. 1C illustrates an example embodiment of a sleeping bag in which the technology, described herein, may be implemented.

FIG. 1D illustrates an example embodiment of a vest in which the technology, described herein, may be implemented.

FIG. 2A illustrates an alternative example embodiment of a pillow according to the technology described herein.

FIG. 2B illustrates another alternative embodiment of a pillow in which the technology described herein may be implemented.

FIG. 3 illustrates an alternative example sachet in which the technology, described herein, may be implemented.

### DETAILED DESCRIPTION

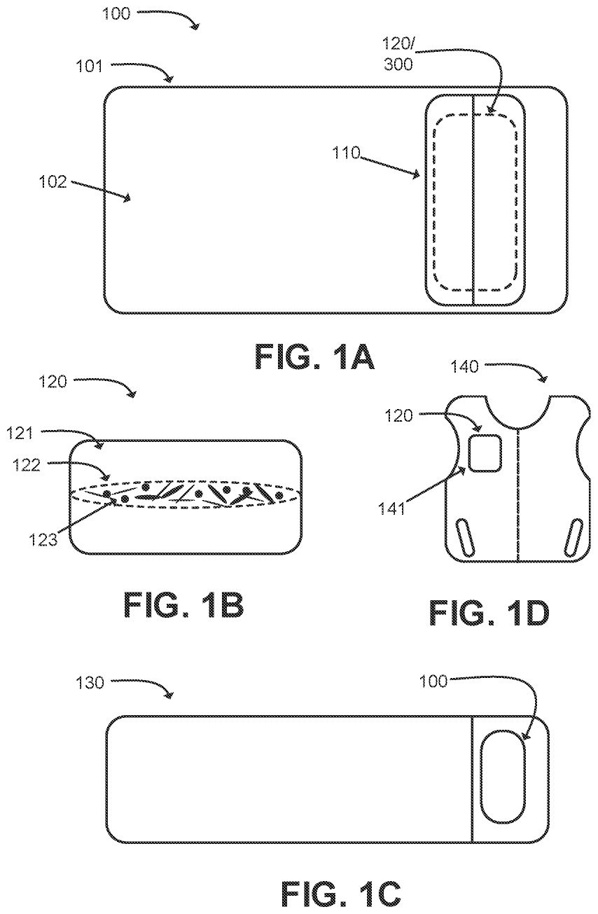

The apparatus, systems, methods, technologies and/or techniques (hereinafter “technology”) described herein may provide a pillow that includes an herbal mixture which is used to provide aromatics and their associated benefit to a user. The technology is described with reference to the example embodiment depicted in FIGS. 1A-3. FIGS. 1A-3 are attached hereto and incorporated herein by reference. The following detailed description refers to the accompanying FIGS. 1A-3. The embodiments depicted in FIGS. 1A-3 are example embodiments only. The present technology may be embodied in many different embodiments as may be envisioned by a person of ordinary skill in the art as a result of reviewing this specification.

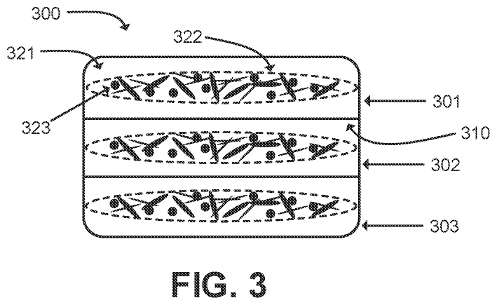

FIG. 1A depicts a non-limiting example environment in which the technology described herein may be implemented. As shown in FIG. 1A, a pillow 100 may include a liner 101 (or shell), fill 102, an envelope 110 and a sachet 120 (or sachet 300, which will be described in reference to FIG. 3). The sachet may include an herbal mixture that may have a sedative effect to a user who lays on the pillow, is near the pillow or otherwise is able to smell the herbal mixture. The pillow 100 used in connection with the technology described herein may be formed from a single component and/or multiple components in a wide variety of structural designs and/or arrangements. FIG. 1A is merely an example embodiment in which the technology described herein may be implemented. While pillow 100 is depicted in FIG. 1A to have a single sachet and envelope, other pillows anticipated by the present disclosure may have multiple sachets and/or envelope (i.e. 2, 3, 4, etc) and each envelope may include more than one sachet.

Pillow 100 may be any size and/or shape of pillow. For example, pillow 100 may be a typical pillow used on a bed, such as a standard pillow (20″×26″), a super standard pillow (20″×28″), a queen pillow (20″×30″), a king pillow (20″×36″), a European square pillow (26″×26″), a neck bolster (18″×6″), etc. Other sizes may be manufactured, such as a small pillow (26″×36″), medium pillow (28″×36″), large (28″×40″) or extra large (32″×44″). The quotation marks following the foregoing dimensions are used to indicated that the dimension is in inches; however, pillow may be manufactured in any dimension and in a wide variety of shapes (i.e. circular, oval, square, rectangular, etc.). Additionally, or alternatively, pillow 100 may be another type of pillow, such as a throw pillow, travel pillow, pregnancy pillow, a wedge, etc. In one embodiment, pillow 100 is an eye pillow that is sized to fit over one eye or both eyes of a user. In this configuration, the eye pillow may be heated or cooled before being applied to the eyes, and the heat/cold coupled with the fragrance from sachet may soothe tired or puffy eyes or eyes that are inflamed from allergies. While pillow 100 is depicted as a standard pillow, the disclosure herein is not limited to any particular type of pillow and may be any type of pillow, or pillow-type object, known in the art. For example, in one embodiment depicted in FIG. 1C, pillow 100 is integrated (sewn, placed in a compartment of sleeping bag, etc.) into a sleeping bag 130 (i.e. a sleeping bag with an integral pillow). In another embodiment, pillow 100 is in the form of an animal bed (i.e. a dog bed, etc. that contains a sachet).

Pillow 100 may be formed from a variety of materials. Typically, pillow is a two-piece design that comprises a fill 102 and a liner 101. The liner 101 is typically an outer lining or enclosure in which the fill the located. The liner 101 is composed of material that forms an enclosure and that generally provides the shape of the pillow. The fill 102, which is also referred to as stuffing or filling, is used to fill the liner 101 to provide the thickness, and density to pillow 100. Fill 102 is used to support a user when pillow 100 is used, such as when a user lays on pillow 100, etc. The fill 102 supports the user by absorbing the force exerted on the pillow 100 by the user, such as by compressing, deforming, etc., in a way that comfortably supports the user. Fill 102 may be any filler material known in the art (e.g. feathers, foam, memory foam, latex, wool (sheep's wool, alpaca, etc.), synthetics, cotton, buckwheat hull, microbeads, kapok, etc.), and the liner 101 may be any type of material known for making shells (e.g. cotton, polyester, blends, wool, etc.). In one embodiment, the fill 102 and liner 101 are made of the same material (e.g. alpaca, cotton, polyester, etc.). In another embodiment, the fill 102 and liner 101 are made from different materials. In other embodiments, pillow 100 may be primarily a single-piece design, such as a single piece of memory foam to which envelope is connected. In other embodiments, pillow 100 may be a three or more-piece design. Pillow 100 may be made in a variety of shapes and have a variety of features. For example, pillow 100 may include a zipper that allows the liner 101 or shell to be separated from the fill 102 (i.e. for washing, etc.). In another embodiment, pillow 100 is sold without fill 102 (i.e. the liner, envelope 110 and/or sachet 120), which allows a user to use any fill and/or insert the user desires in pillow 100. Users may cover pillow 100 with, for instance, a pillowcase (not shown).

Envelope 110 may be connected to liner 101 and may provide an enclosure (or a second enclosure, when the enclosure formed by liner is the first enclosure) (e.g. a pocket, a compartment, etc.) in which sachet 120 may reside. In the embodiment depicted in FIGS. 1A and 1B, the envelope 110 may be connected to an inner surface of liner 101 (i.e. within the enclosure formed by the liner). However, in other embodiments, the envelope 110 may be connected to an outer surface of the liner 101. Envelope 110 may be configured to house sachet 120 in the enclosure by being a size that permits sachet 120 to be placed in envelope 110. Envelope 110 may be formed as part of the liner, may be attached to liner 101 (i.e. sewn, hook and loop attachments, zippers, etc.). In the embodiment depicted in FIG. 1A, two overlapping flaps (i.e. two sheets, in a variety of sizes depending on the size of sachet 120) of material (i.e the same or different material as shell) are sewn to liner 101 (i.e. using liner as part of the enclosure). In this embodiment, the overlapping material of envelope 110 and the portion of the liner 101 adjacent to the flaps of material form envelope 110. While envelope is depicted as overlapping flaps to enclose sachet 120, a zippered enclosure (or an enclosure with overlapping flaps) that does not rely on liner 101 to form the enclosure may be, for instance sewn on liner 101, etc. Envelope 110 may be used to house, or store, sachet 120 and/or to permit sachet 120 to be removed (i.e. to change sachet 120, to wash all or part of pillow (i.e. the liner, the liner and envelope 110, etc.)). Envelope 110 may be made of the same and/or different material as liner 101.

Sachet 120 may be a herb-filled container (i.e. bag, packet, etc.) that is used to provide the aroma of the herbs to the user of pillow 100. Sachet 120 may include a shell that may be formed from any of the materials used for liner 101 or different materials. The materials used to form sachet shell 121 may be selected to allow the aroma of herbs to easily disperse through sachet shell 121 while not being porous enough to permit the herbs within sachet 120 to escape. As shown in FIG. 1B, sachet 120 may include a sachet shell 121 or shell 121 (sachet shell is hereinafter shell 121 or sachet shell 121, as context makes clear), a sachet closure 122, or closure 122, and an herbal mixture 123. The sachet 120 used in connection with the technology described herein may be formed in a variety of different designs and may have fewer and/or additional components than reflected in FIG. 1B. The sachet 120 reflected in FIG. 1B is merely an example embodiment in which the technology described herein may be implemented. Many other embodiments of sachet 120 are anticipated by the disclosure herein, such as sachet 300. For example, sachet 120 may be in the form of a disposable sachet that does not include a zipper but that contains the herbal mixture in a shell (i.e. a shell that may be discarded, such as a biodegradable shell, etc.).

Sachet shell 121, or shell 121 may be a material that envelopes or contains herbal mixture 123. Shell 121 may be formed of a material that is breathable or that otherwise allows the aromas that emanate from herbal mixture 123 to be dispersed to the user of pillow 100. For example, shell 121 may be comprised of a fabric (e.g. cotton, wool, alpaca, polyester, nylon, a blend of any of the foregoing etc.) through which the aromas of herbal mixture 123 may disperse from pillow 100 to the user (i.e. through shell 121, envelope 110, and/or other portions of pillow, to the user). Sachet 120 may include a closure 122, which may correspond to a zipper other sealable or resealable opening (i.e. folded over material, etc.), that may allow the herbal mixture to be inserted into shell 121 and/or removed from shell 121 (i.e. to allow herbal mixture 123 to be changed, initially inserted, removed, etc.). In other embodiments (such as disposable sachets), no closure 122 exists, and the sachet shell 121 may be glued, sewn or otherwise sealed to provide the enclosure in which herbal mixture 123 is located.

Herbal mixture 123 may be comprised of one or more herbs that are desirable for use in pillow. In one embodiment, herbal mixture 123 may be comprised of one or more types of hops (admiral hops, angus hops, atlas hops . . . Zappa hops, Zenith hops, Zythos hops, etc.). herbal mixture 123 may further include other types of aromatics, such as lavender, jasmine, chamomile, lemongrass, rose petals, mugwort, mint, etc. In one embodiment, herbal mixture 123 includes lavender, jasmine, chamomile, lemongrass and rose petals. In another embodiment, herbal mixture includes hops, mugwort, lavender, jasmine, chamomile, lemongrass and rose petals. The varieties of herbal mixture 123 may vary greatly and may be chosen based on the desired effect, and the disclosure herein should not be construed to be limited to the particular herbal mixtures 123 disclosed as examples herein. In a first example herbal mixture 123 that promotes sleep, the herbal mixture 123 may include one or more hops and a variety of aromatics, including mugwort, jasmine, chamomile, lavender, rose petals, lemongrass and mint. In a second example embodiment of herbal mixture 123, such as may be used in a pet bed, herbal mixture 123 may include cedar, rosemary and/or lavender.

In light of the foregoing description, many other embodiments of the technology described herein are evident to a person having ordinary skill in the art. For example, and not limitation, FIG. 1C depicts an example embodiment of a sleeping bag in which the technology, described herein, may be implemented. Sleeping bag 130 may include pillow 100, such as by being connected to pillow (i.e. sewn, Velcro, zippered enclosure, etc.). As further example, FIG. 1D reflects a vest 140 having a pocket 141 in which a sachet 120 may be disposed. The sachet 120 may include herbal mixture 123 that may satisfy a want or need of the wearer of the vest. For example, and not limitation, the sachet 120 in pocket 141 may have an herbal mixture 123 that induces alertness, focus, relaxation, imagination, clarity, contentment, etc. The components illustrated in FIGS. 1A-1D are provided for explanatory purposes only, and the disclosure herein is not intended to be limited to, or to require, the components provided therein or the embodiments depicted in the figures. Pillows anticipated by the present disclosure may include additional components, fewer components, different components, and/or differently arranged or designed components than illustrated in FIGS. 1A-1D. Also, in some implementations, one or more of the components of the pillows anticipated by the present disclosure may perform one or more functions described as being performed by another one or more of the components of such pillows.

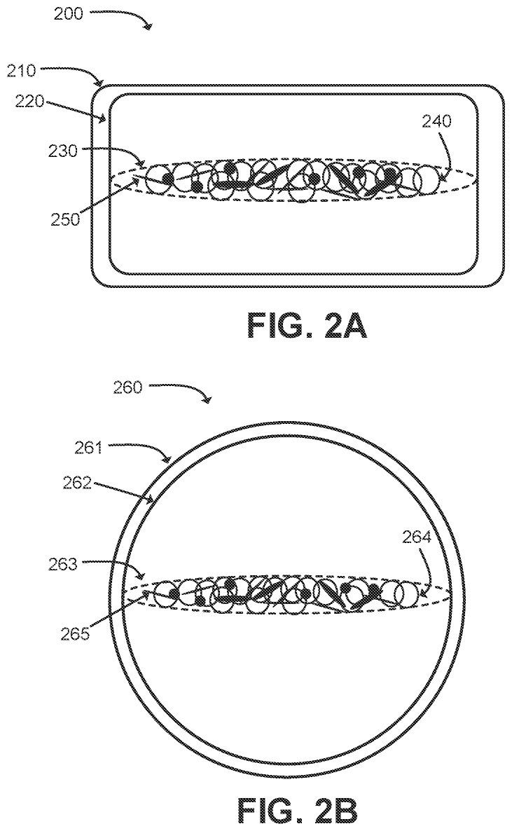

FIG. 2A illustrates an alternative example embodiment of a pillow according to the technology described herein. Pillow 200 includes a pillowcase 210 and a liner 220. Liner 220 includes a closure 230, fill 240 and herbal mixture 250. Pillow 200 is provided for explanatory purposes only, and the disclosure herein is not intended to be limited to, or to require, the components provided in connection with pillow 200 or the embodiment depicted in FIG. 2A. Pillows anticipated by the present disclosure may include additional components, fewer components, different components, and/or differently arranged or designed components than illustrated in FIG. 2A. Also, in some implementations, one or more of the components of pillow 200 may perform one or more functions described as being performed by another one or more of the components of pillow 200.

Pillow 200 may be similar to pillow 100 except that pillow 200 may not include a sachet within an envelope. Instead, pillow 200 may include herbal mixture 250 within liner 220 along with fill 240. Herbal mixture 250 may correspond to any of the embodiments of herbal mixture 123 described herein. Fill 240 may be any of the fill materials described herein, including feathers, inserts, foam, memory foam, latex, wool (sheep's wool, alpaca, etc.), synthetics, cotton, buckwheat hull, microbeads, kapok, etc.), including those materials described herein in relation to fill 102. As depicted in FIG. 2A, fill 240 is alpaca wool. Liner 220 may be any type of material known for making liners or shells of pillows (e.g. cotton, polyester, blends, wool, etc.), including those described for liner 101. Similarly, pillowcase 210 may be formed from any type of material used for pillowcases (e.g. cotton, polyester, blends, wool, canvas, etc.). Closure 230 may be a zipper, sewn closure, or other sealable and/or resealable closure that allows fill 240 and herbal mixture 250 to be added to, maintained within and/or removed from pillow 200. For example, and not limitation, closure 230 may be any of the embodiments described herein in relation to closure 122. Pillow 200 may be used in a variety of ways, including those described herein. Additionally, or alternatively, pillow 200 may be an 8″×8″ embodiment that is placed in a variety of areas (i.e. clothing drawers, etc.) to transfer aromas to items nearby (i.e. clothes, pajamas, lingerie, sheets, etc.) by allowing the items to receive the scent of herbal mixture 250. In another embodiment, pillow 200 is a neck bolster pillow.

FIG. 2B illustrates another alternative embodiment of a pillow in which the technology described herein may be implemented. Pillow 260 may be the same as pillow 200 except that pillow 260 is a round shape, which may be used as, for example a dog bed. Pillow 260 includes a pillowcase 261, a liner 262, a closure 263, fill 264 and herbal mixture 265. These items may be the same as, or similar to, the similarly identified components of pillow 100 and/or pillow 200. When used as a dog bed, pillow 260 may include a pillowcase 261 made from canvas, and herbal mixture 265 may include rosemary, cedar, lemongrass, and thyme. In the embodiment depicted in FIG. 2B, pillow 260 includes fill 264 that is in liner 262 along with herbal mixture 265. In other embodiments, herbal mixture 265 is located within a sachet that is either placed inside of liner 262 or within an envelope, like liner 110, that is connected to liner.

FIG. 3 illustrates an alternative example sachet in which the technology, described herein, may be implemented. Sachet 300 may be the same as, or similar to, sachet 120 except that sachet 300 has at least two compartments. In the embodiment depicted in FIG. 3, sachet 300 includes a first compartment 301, a second compartment 302, and a third compartment 303 (collectively the “compartments” and each a “compartment”). Each of the compartments may comprise a shell 321 (or sachet shell 301), a closure 322 and an herbal mixture 323. Sachet 300 is provided for explanatory purposes only, and the disclosure herein is not intended to be limited to, or to require, the components provided in connection with sachet 300 or the embodiment depicted in FIG. 3. Sachets anticipated by the present disclosure may include additional components, fewer components, different components, and/or differently arranged or designed components than illustrated in FIG. 3. Also, in some implementations, one or more of the components of sachet 300 may perform one or more functions described as being performed by another one or more of the components of sachet 300.

First compartment 301, second compartment 302 and third compartment 303 may be separate enclosures of herbal mixture 323, each of which is similar to sachet 120 but are joined together to form sachet 300 by joint 310. Each of the compartments may be formed from sachet shell 321, which may be the same material or separate materials. For example, the compartments may be formed from the same shell 321 when joint 310 is formed by sewing, gluing, or otherwise separating the shell 321 into the compartments. In another embodiment, shell 321 is different for each material, making each compartment a separate sachet which are joined together by joint 310 to form sachet 300. In this embodiment, each compartment is joined by joint 310 to the adjacent compartment, and joint 310 again corresponds to a stitched seam, a glued same, etc. Sachet shell 321, or shell 321 may be a material that envelopes or contains herbal mixture 323, similar to shell 121. Shell 321 may be formed of a material that is breathable or that otherwise allows the aromas that emanate from herbal mixture 323 to be dispersed to the user of a pillow that includes sachet, such as pillow 100. Shell 321 may be the same as, or similar to shell 121 except that shell 321 may be used to form more than one (or just one) compartment. Each compartment may include a closure 322, which may correspond to a zipper other sealable or resealable opening (i.e. folded over material, etc.), that may allow the herbal mixture to be inserted into shell 321 and/or removed from shell 321 (i.e. to allow herbal mixture 323 to be changed, initially inserted, removed, etc.). In other embodiments (such as disposable sachets), no closure 322 exists, and the sachet shell 321 for each compartment may be glued, sewn or otherwise sealed to provide the enclosure in which herbal mixture 323 is located. Herbal mixture 323 may be the same as, or similar to, herbal mixture 123. Each compartment of pillow 300 may include the same herbal mixture 323 or a different herbal mixture 323 (different herbal materials or mixtures of herbal materials described herein), depending on the needs of the user of the pillow in which sachet 300 is placed.

The foregoing description provides illustration and description, but is not intended to be exhaustive or to limit the implementations to the precise embodiments disclosed herein. Modifications and variations are possible in light of the above disclosure or may be acquired from practice of the embodiments. It will be apparent that the apparatus, systems, methods, technologies and/or techniques, as described above may be implemented in many different forms of implementations in addition to those illustrated in the Figures. The actual hardware and/or materials used to implement the apparatus, systems, methods, technologies and/or techniques is not limited to the embodiments depicted in the Figures or described above.

It should be emphasized that the terms comprises/comprising when used in the specification are taken to specify the presence of stated features, integers, steps or components but does not prevent the presence or addition of additional features, integers, steps, components, or other groups thereof.

Even though particular combinations of features are recited in the specification, these combinations are not intended to limit the disclosure of the embodiments. In fact, many of these features may be combined in ways not specifically recited in the disclosure. No element, act, or instruction used in the present application should be construed as critical or essential to the embodiments unless explicitly described as such. Also, as used herein, the articles “a” and “and” are intended to include one or more items and may be used interchangeably with one or more. Where only one item is intended, the term “one” or similar language is used. Further, the phrase “based on” is intended to mean “based, at least in part, on” unless explicitly stated otherwise.

What is claimed is:

1. A pillow comprising:
a liner that forms an enclosure;
a fill that is located within the enclosure of the liner, the fill deforming to provide support when the pillow is used;
an herbal mixture, located within the enclosure of the liner, the herbal mixture providing an aroma to the user when the pillow is used.

2. The pillow of claim 1, where the herbal mixture and fill are mixed together within the enclosure of the liner.

3. The pillow of claim 1 further comprising:
an envelope connected to an inner surface of the liner, the envelope providing a second enclosure;
a sachet located within the second enclosure, the herbal mixture located within the sachet.

4. The pillow of claim 3, where the envelope is connected to an inner surface of the liner.

5. The pillow of claim 3, where the envelope is formed from two flaps of material and a portion of the liner adjacent to the two flaps of material.

6. The pillow of claim 5, where the envelope includes a closure.

7. The pillow of claim 3, where the sachet includes a sachet shell configured to allow the aroma from the herbal mixture to be dispersed from the sachet while maintaining the herbal mixture within the sachet.

8. The pillow of claim 3, where the sachet includes a sachet closure.

9. The pillow of claim 1 further including a pillowcase, the liner being disposed within the pillowcase.

10. The pillow of claim 1, where the herbal mixture includes lavender, jasmine, chamomile, lemongrass and rose petals.

11. The pillow of claim 1, where the herbal mixture includes hops and at least one of mugwort, jasmine, chamomile, lavender, rose petals, lemongrass or mint.

12. The pillow of claim 1, where the herbal mixture includes cedar, rosemary and lavender.

13. The pillow of claim 1, where the herbal mixture includes cedar, rosemary, lemongrass and thyme.

14. The pillow of claim 13, where the liner is formed in a circular shape.

15. The pillow of claim 3 where the sachet is disposable.

16. The pillow of claim 14 further including a pillowcase formed from canvas, the liner being disposed within the pillowcase.

17. The pillow of claim 1 where the liner is formed in the shape of a neck bolster.

18. The pillow of claim 3, wherein the sachet has at least two compartments, each compartment including the herbal mixture.

19. The pillow of claim 18, wherein the sachet has exactly three compartments.

20. The pillow of claim 18, wherein the herbal mixture in each compartment is composed of the same materials.
